(19) United States
(12) Patent Application Publication
(10) Pub. No.: US 2008/0254146 A1
(43) Pub. Date: Oct. 16, 2008

(54) METHOD OF PROVIDING HEMOSTASIS IN ANTI-COAGULATED BLOOD

(75) Inventors: Raymond J. Huey, Orange, CT (US); Giacomo Basadonna, Haddam, CT (US)

Correspondence Address: MICHAUD-DUFFY GROUP LLP, 306 INDUSTRIAL PARK ROAD, SUITE 206, MIDDLETOWN, CT, 06457, US

(73) Assignee: Z-MEDICA CORPORATION, Wallingford, CT (US)

(21) Appl. No.: 12/101,336

(22) Filed: Apr. 11, 2008

Related U.S. Application Data
(60) Provisional application No. 60/923,416, filed on Apr. 13, 2007.

(51) Int. Cl. A61K 33/06; A61P 7/04

(52) U.S. Cl. 424/684

(57) ABSTRACT

A method of clotting blood includes the step of administering a therapeutically effective amount of a composition comprising zeolite as the active ingredient to a wound from which the blood emanates. A method of arresting blood flowing from a wound includes the steps of providing a patient being inflicted with a bleeding wound and administering a therapeutically effective amount of a composition comprising zeolite as the active ingredient to the bleeding wound. A method of facilitating the formation of blood clots includes the step of contacting blood with a negatively charged surface wherein upon contacting the blood with the negatively charged surface a clotting mechanism is initiated. In any of the foregoing methods, the blood has a compromised ability to form clots. The blood may be from a person diagnosed with hemophilia or von Willebrand disease.

### CROSS REFERENCE TO RELATED APPLICATION

This application claims the benefits of U.S. Provisional Patent Application Ser. No. 60/923,416, filed on Apr. 13, 2007, the contents of which are incorporated by reference herein in their entirety.

### TECHNICAL FIELD

The present invention relates generally to methods of providing hemostasis in blood that is resistant to normal clotting functions and, more particularly, to methods of providing hemostasis in patients having compromised blood clotting functions due to the use of anticoagulant compositions or due to deficiencies in factors that contribute to clotting abilities.

### BACKGROUND OF THE PRESENT INVENTION

Blood is a liquid tissue that includes red cells, white cells, corpuscles, and platelets dispersed in a liquid phase. The liquid phase is plasma, which includes acids, lipids, solublized electrolytes, and proteins. Some proteins and other substances in the plasma are collectively known as clotting factors (indicated by Roman numerals) and function together to promote the coagulation of blood. The proteins are suspended in the liquid phase. One particular protein suspended in the liquid phase is fibrinogen.

Anticoagulant drugs are typically prescribed to individuals with increased tendencies for thrombosis, which is the formation of clots in the blood, or as prophylaxis in individuals who have pre-existing blood clots to reduce the risks of embolism. These drugs are also indicated for the long-term anticoagulation treatment of patients having certain kinds of surgery, heart disease, following stent placement, valve replacement, atrial fibrillation, and the like.

One such anticoagulant drug is warfarin, which is a synthetic derivative of 4-hydroxycoumarin and which decreases the natural abilities of blood to coagulate by interfering with the hepatic synthesis of vitamin K-dependent clotting factors, particularly those indicated as Factors II, VII, IX, and X. It also interferes with the regulatory factors protein C, protein S, and protein Z. Other proteins not involved in blood clotting such as osteocalcin and matrix Gla protein may also be affected.

Warfarin is typically used by individuals suffering from atrial fibrillation to reduce the incidence of stroke, thromboembolism, complications associated with cardiac valve replacement, myocardial infarction, and the like. The degree of anticoagulation in an individual undergoing warfarin therapy is determined by the international normalized ratio (INR) of the blood. A normal INR range is 0.8 to 1.2, whereas individuals taking warfarin typically have an INR target range of 2.0 to 3.0. These individuals generally have difficulty in achieving hemostasis after experiencing a wound resulting from trauma (e.g., from an accident or a medical procedure).

Several adverse effects have been noted with regard to individuals undergoing warfarin therapy. Such adverse effects include, but are not limited to, paresthia, headache, joint and/or muscle pain, shortness of breath, swelling, weakness, hypotension, jaundice, fever, hepatitis, alopecia, elevation of liver enzymes, and hemorrhage (bleeding). Hemorrhage is the most common and dangerous complication associated with the regular use of warfarin and occurs in about 2% to about 5% of treated patients with a significant increase in hospitalization and associated costs. The hemorrhage may be from any tissue or organ and may be fatal or non-fatal. Hemorrhage can also be exacerbated by certain vascular defects, abnormalities in the blood, or deficiencies of one or more of the coagulation factors.

Hemorrhage can also occur as the result of traumatic injury irrespective of whether or not the hemorrhaging individual is undergoing warfarin therapy. When a hemorrhage occurs as a result of trauma and the blood is normal (i.e., not significantly deficient in any component that would alter its ability to clot or not subject to anticoagulant drugs), hemostasis is initiated normally. Hemostasis is the arrest of blood flow from an injured blood vessel and requires the combined functions of the vascular, platelet, and plasma factors. In initiating hemostasis in response to trauma, the physiologic process of thrombosis begins. In thrombosis, the platelets aggregate and/or the fibrinogen reacts with water and thrombin (an enzyme) to form fibrin, which is insoluble in blood and which polymerizes to form the clots.

When hemorrhage occurs as the result of traumatic injury in individuals undergoing warfarin therapy (or taking some other anticoagulating drug), the ability of the blood to experience normal clotting functions is compromised. This lack of normal clotting functions may prove to be problematic during the course of an attempted emergency treatment of the individual. For example, a caregiver at an accident scene may be unaware that an injured individual may be undergoing warfarin therapy and may attempt to provide normal medical treatment, the effects of which may have limited efficacy due to the individual's lack of clotting ability. Treatment of an individual known to be undergoing warfarin therapy via planned surgery, on the other hand, may be less problematic but still pose problems for the persons performing the surgery because the effect of warfarin has to be reversed.

Hemorrhage can also occur as the result of hemophilia. Hemophilia is the name for several hereditary genetic illnesses that impair the ability of a body to control bleeding. Various types of hemophilia exist. Hemophilia A, the most common form of hemophilia, is a blood clotting disorder caused by a mutation of the Factor VIII gene, which leads to a deficiency in Factor VIII. Inheritance is X-linked recessive; thus, males are affected (1 in 10,000) while females are carriers or very rarely display a mild phenotype. Hemophilia B, the second most common form, is a blood clotting disorder caused by a mutation of the Factor IX gene, which may indicate a deficiency in Factor IX. Hemophilia (all types) affects about 18,000 people in the United States. Each year, about 400 babies are born with the disorder. Patients with hemophilia may bleed for a longer time than others after an injury or accident. They also may bleed internally, especially in the joints (knees, ankles, and elbows).

Hemorrhage can also occur as the result of von Willebrand disease. Von Willebrand disease is the most common hereditary coagulation abnormality described in humans, although it can also be acquired as a result of other medical conditions. It arises from a qualitative or quantitative deficiency of von Willebrand factor (vWF), a multimeric protein that is required for platelet adhesion. The vWF factor is present in blood plasma and produced constitutively in endothelium (in the Weibel-Palade bodies), megakaryocytes (α-granules of platelets), and subendothelial connective tissue. Von Willebrand factor is not an enzyme and therefore has no catalytic activity. Its primary function is binding to other proteins, particularly Factor VIII, and it is important in platelet adhesion to wound sites.

Von Willebrand factor binds to cells and molecules in a number of different scenarios. These scenarios include, but are not limited to: (a) Factor VIII is bound to vWF whilst inactive in circulation, the Factor VIII degrades rapidly when not bound to vWF, and the Factor VIII is released from vWF by the action of thrombin; (b) vWF binds to collagen, e.g., when it is exposed in endothelial cells due to damage occurring to the blood vessel; (c) vWF binds to platelet gpIb when it forms a complex with gpIX and gpV (occurs under all circumstances, but is most efficient under high shear stress (i.e., rapid blood flow in narrow blood vessel)); and (d) vWF binds to other platelet receptors when they are activated, e.g., by thrombin (i.e., when coagulation has been stimulated).

There are three types of hereditary von Willebrand disease, namely, Types I, II, and III. Types I and II are considered herein to be mild. In the mild form, a ristocetin co-factor is decreased and different levels of von Willebrand disease multimers are depleted. Type III is considered herein to be severe. In severe von Willebrand disease, only less than 10% expression of factor VIII is present and no detectable level of von Willebrand factor is present.

The various types of von Willebrand disease present varying degrees of bleeding tendency. In any form, bruising, nosebleeds, heavy menstrual periods (in women), and blood loss during childbirth (which is rare) may occur. Also, internal bleeding or joint bleeding may also occur. This type of bleeding is generally only in the severe form of von Willebrand disease and is rare. Particularly with regard to the severe form, death may occur.

Based on the foregoing, it is a general object of the present invention to provide methods of facilitating hemostasis in individuals undergoing warfarin therapy or being deficient in certain clotting factors that overcome or improve upon the prior art, such methods being in response to trauma sustained either as a result of an accident or an intentionally inflicted wound.

### SUMMARY OF THE PRESENT INVENTION

In one aspect, the present invention is directed to a method of clotting blood. The blood exhibits a reduced tendency to clot (compared to normal blood) and may be from a person undergoing an anticoagulant therapy or having type A or B hemophilia or von Willebrand disease. In the method a therapeutically effective amount of a composition comprising zeolite as the active ingredient is administered to a wound from which the blood emanates. Upon contacting the blood, the zeolite causes the blood to clot.

In another aspect, the present invention is directed to a method of arresting blood flowing from a wound. The method comprises the step of administering a therapeutically effective amount of a composition comprising zeolite as the active ingredient to the bleeding wound. The blood has a reduced tendency to clot (compared to normal blood) may be from a person undergoing an anticoagulant therapy or having at least one of hemophilia A or B or von Willebrand disease.

In another aspect, the present invention is directed to a method of facilitating the formation of blood clots. In the method, blood treated with an anticoagulant composition, being deficient in either Factor VIII or Factor IX, and/or being deficient in von Willebrand factor is provided and contacted with a negatively charged surface. Upon contacting the blood with the negatively charged surface, a clotting mechanism is initiated.

### DETAILED DESCRIPTION OF THE PREFERRED EMBODIMENT

Disclosed herein are methods for delivering hemostatic materials to interface regions of tissue and blood vessels to promote the clotting of blood and to limit the degree of bleeding in individuals having coagulation disorders. As used herein, the term “coagulation disorder” refers to an inability or reduced ability of blood to produce clots. The methods generally comprise stopping bleeding that results from trauma (e.g., from unintentional wounds as well as intentional wounds such as those resulting from surgical procedures) to tissue or organs in individuals undergoing anticoagulant drug therapy. Anticoagulant drugs with which the methods described herein may be used include, but are not limited to, warfarin and other derivatives of 4-hydroxycoumarin. One exemplary hemostatic material that can be used with the methods of the present invention is zeolite.

As used herein, the term “zeolite” refers to a crystalline form of aluminosilicate having the ability to be dehydrated without experiencing significant changes in the crystalline structure. The zeolite typically includes one or more ionic species such as, for example, calcium and sodium moieties. In zeolites containing calcium and sodium, the calcium portion contains crystals that are about 5 angstroms in size, and the sodium portion contains crystals that are about 4 angstroms in size. The preferred molecular structure of the zeolite is an “A-type” crystal, namely, one having a cubic crystalline structure that defines round or substantially round openings. In its original state, zeolite is negatively charged, which means it has a propensity for attracting positively charged ions.

Zeolites for use in the disclosed applications may be naturally occurring or synthetically produced. Numerous varieties of naturally occurring zeolites are found as deposits in sedimentary environments as well as in other places. Naturally occurring zeolites that may be applicable to the compositions described herein include, but are not limited to, analcite, chabazite, heulandite, natrolite, stilbite, and thomosonite. Synthetically produced zeolites that may also find use in the compositions and methods described herein are generally produced by processes in which rare earth oxides are substituted by silicates, alumina, or alumina in combination with alkali or alkaline earth metal oxides.

The zeolite may be mixed with or otherwise used in conjunction with other materials. These materials may be used as fillers or inert ingredients with the zeolite. Preferably, these materials have the ability to be dehydrated without significant changes in crystalline structure. Such materials include, but are not limited to, magnesium sulfate, sodium metaphosphate, calcium chloride, dextrin, polysaccharides, combinations of the foregoing materials, and hydrates of the foregoing materials. Clays, diatomaceous earth, bioactive glass, chitosan, polymeric materials, and combinations of the foregoing may also be mixed with the zeolite. The present invention is not limited in this regard, however, as other materials may be used in conjunction with the zeolite.

The zeolite may be administered in any suitable form. Suitable forms include, but are not limited to, particles, beads, pellets, chips, flakes, powders, pastes, gels, combinations of the foregoing, and the like.

In effecting hemostasis at a wound site in an individual undergoing warfarin therapy (or in any individual having a coagulation disorder), the zeolite is administered in a therapeutically effective amount utilizing any suitable delivery mechanism. A therapeutically effective amount is any amount that is capable of causing the anticoagulated blood of the individual to sufficiently clot. If the zeolite is in the form of loose particles such as pellets, beads, or the like, the zeolite can be poured or otherwise placed directly onto the wound site. Loose powder having sufficient fluidity can also be poured or placed directly onto the wound site. If the zeolite is in the form of a paste, e.g., suspended in a gel carrier, the zeolite can be spread or smeared topically over the wound, or it can be applied to bandages, gauze, pads, or other like materials and used to dress the wound. Furthermore, sponges and cloths into which the zeolite is impregnated or otherwise incorporated may be applied to or even packed into the wound.

In each of the Examples provided below, human plasma was obtained from two or more patients affected by one single studied condition. The human plasma was obtained from George King Bio-Medical, Inc., Overland Park, Kans. In each of the Examples, results are shown as a mean plus or minus the standard deviation. Student t test was performed as statistical analysis and p<0.05 was considered as significant.

### EXAMPLE 1

### Use of Zeolite to Treat Human Plasma from Patients Undergoing Warfarin Therapy

Human plasma was obtained from patients treated with Coumadin® (a brand of warfarin) and having INR levels of 1.9, 3.6, and 5.3. Plasma from three patients per INR level was analyzed. The plasma was divided in 2 groups (Control and Study 1) and was tested in vitro in a modified PT manual test. For the test, 0.25 ml of plasma was incubated with 25% dilution in 0.9% saline of Simplastin Excel (thromboplastin reagent, available from Biomerieux, Durham, N.C.). Zeolite material was added to the Study 1 group samples. Results are shown in Table 1.

TABLE 1
Table 1: Zeolite-treated plasma clots significantly faster than
untreated controls.

| | Time to clot: |
|---|---|
| | seconds |
| INR 1.9 | |
| Control n = 2 | 172.5 ± 10.6 |
| Study 1 n = 7 | 128.6 ± 19.1* |
| INR 3.6 | |
| Control n = 4 | 596.3 ± 39.4 |
| Study 1 n = 8 | 238.1 ± 87.6** |
| INR 5.3 | |
| Control n = 4 | 311.3 ± 83.4 |
| Study 1 n = 8 | 175 ± 21.2*** |

*p < 0.001
**p < 0.0001
***p < 0.04

Human plasma treated with zeolite clotted significantly faster than untreated control plasma independently from the INR level.

### EXAMPLE 2

### Use of Zeolite to Treat Human Plasma from Patients Having Hemophilia

Human plasma was also obtained from patients diagnosed with Hemophilia A (Factor VIII less than 1%) and Hemophilia B (Factor IX less than 1%). This human plasma was divided into 2 groups (Control and Study 2) and was tested in a modified APTT manual test. In this test, 0.25 ml of plasma was incubated at 37 C in the presence of 0.025 M CaCl (0.25 ml obtained from Biomerieux, Durham, N.C.) and 0.25 ml Platelet Factor 3 reagent (Partial Thromboplastin) (also obtained from Biomerieux, Durham, N.C.). Zeolite material was added to the Study 2 group samples. Results are shown in Table 2.

TABLE 2
Table 2: Zeolite treated plasma clots significantly faster than
untreated controls.

| | Time to clot: |
|---|---|
| | seconds |
| Hemophilia A | |
| Control n = 15 | 133.8 ± 26.9 |
| Study 2 n = 26 | 106.7 ± 22.1* |
| Hemophilia B | |
| Control n = 12 | 105.2 ± 32.2 |
| Study 2 n = 18 | 84.2 ± 23.2** |

*p < 0.002
**p < 0.05

Human plasma treated with zeolite clotted significantly faster than untreated control plasma for both Hemophilia A and B.

### EXAMPLE 3

### Use of Zeolite to Treat Human Plasma from Patients Having Von Willebrand Disease

Human plasma was obtained from patients affected by von Willebrand disease, both mild (Type I and II) and severe (Type III). The human plasma was divided into 2 groups (Control and Study 3) and was tested in a modified APTT manual test. For this test, 0.25 ml of plasma was incubated at 37 C in the presence of 0.025 M CaCl (0.25 ml obtained from Biomerieux, Durham, N.C.) and 0.25 ml Platelet Factor 3 reagent (Partial Thromboplastin) (also obtained from Biomerieux, Durham, N.C.). Zeolite material was added to the Study 3 group samples. Results are shown in Table 3.

TABLE 3
Table 3: Zeolite treated plasma clots significantly faster than
untreated controls.

| | Time to clot: |
|---|---|
| | seconds |
| Mild von Willebrand | |
| Control n = 5 | 83.6 ± 5.5 |
| Study 3 n = 19 | 75.6 ± 5.7* |
| Severe von Willebrand | |
| Control n = 8 | 124.1 ± 15.4 |
| Study 3 n = 13 | 109.5 ± 19.8** |

*p < 0.01
**p < 0.01

Human plasma treated with zeolite clotted significantly faster than untreated control plasma for both forms of von Willebrand disease.

In taking into account the results of each of the above Examples, it can be concluded that zeolite clots human plasma faster than untreated controls in the following conditions: patients treated with Coumadin® (INR 1.9, 3.6, 5.3), patients affected by Hemophilia A (Factor VIII less than about 1%), and patients affected by Hemophilia B (Factor IX less than about 1%). In addition, zeolites clot human plasma faster than untreated controls in patients affected by von Willebrand disease both mild and severe.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A method of clotting blood, said method comprising the step of:
administering a therapeutically effective amount of a composition comprising zeolite as the active ingredient to a wound from which said blood emanates;
wherein said blood has a compromised ability to form clots.

2. The method of claim 1, wherein said blood includes a coumarin-based composition as an anticoagulant composition.

3. The method of claim 1, wherein said blood includes warfarin.

4. The method of claim 1, wherein said blood is obtained from a patient diagnosed with Hemophilia A.

5. The method of claim 1, wherein said blood is obtained from a patient diagnosed with Hemophilia B.

6. The method of claim 1, wherein said blood is obtained from a patient diagnosed with von Willebrand disease.

7. The method of claim 1, wherein said zeolite is selected from the group consisting of analcite, chabazite, heulandite, natrolite, stilbite, and thomosonite.

8. A method of arresting blood flowing from a wound, said method comprising the steps of:
providing a patient being inflicted with a bleeding wound; and
administering a therapeutically effective amount of a composition comprising zeolite as the active ingredient to said bleeding wound;
wherein said blood has a compromised ability to form clots.

9. The method of claim 8, wherein said patient is treated with a coumarin-based composition as an anticoagulant composition.

10. The method of claim 8, wherein said patient is treated with warfarin.

11. The method of claim 8, wherein said blood is obtained from a patient diagnosed with Hemophilia A.

12. The method of claim 8, wherein said blood is obtained from a patient diagnosed with Hemophilia B.

13. The method of claim 8, wherein said blood is obtained from a patient diagnosed with von Willebrand disease.

14. The method of claim 8, wherein said step of administering said therapeutically effective amount of said composition comprises placing said composition directly on said bleeding wound.

15. The method of claim 8, wherein said step of administering said therapeutically effective amount of said composition comprises placing said composition on bandages and placing said bandages directly on said bleeding wound.

16. A method of facilitating the formation of blood clots, said method comprising:
contacting blood with a negatively charged surface;
wherein said blood has a compromised ability to form clots and wherein upon contacting said blood with said negatively charged surface a clotting mechanism is initiated.

17. The method of claim 16, wherein said blood includes warfarin.

18. The method of claim 16, wherein said blood is obtained from a patient diagnosed with Hemophilia A.

19. The method of claim 16, wherein said blood is obtained from a patient diagnosed with Hemophilia B.

20. The method of claim 16, wherein said blood is obtained from a patient diagnosed with von Willebrand disease.

21. The method of claim 16, wherein said blood is provided from a person undergoing warfarin therapy.

22. The method of claim 16, wherein said blood is provided from a person wounded in a surgical procedure.

23. The method of claim 16, wherein said step of contacting said blood with a negatively charged surface comprises administering a therapeutically effective amount of a composition containing a zeolite to said blood.

24. The method of claim 23, wherein said step of administering a therapeutically effective amount of a composition containing a zeolite to said blood comprises placing said composition directly onto a wound from which said blood emanates.
